(19) United States
(12) Patent Application Publication
(10) Pub. No.: US 2026/0033731 A1
(43) Pub. Date: Feb. 5, 2026

(54) HIGH RESOLUTION BLOOD PRESSURE MEASUREMENT

(71) Applicant: Welch Allyn, Inc., Skaneateles Falls, NY (US)

(72) Inventors: David E. Quinn, Moravia, NY (US); Tyson B. Whitaker, Arden, NC (US); Gene J. Wolfe, Pittsford, NY (US)

(21) Appl. No.: 19/283,728

(22) Filed: Jul. 29, 2025

Related U.S. Application Data
(60) Provisional application No. 63/677,477, filed on Jul. 31, 2024.

(51) Int. Cl. A61B 5/0225; A61B 5/0235

(52) U.S. Cl. CPC A61B 5/0225; A61B 5/0235; A61B 2562/0247

(57) ABSTRACT

A method of measuring blood pressure includes estimating systolic and diastolic blood pressures while inflating a cuff. The estimates of the systolic and diastolic blood pressures are based on waveform oscillations of pressure detected from the cuff. First and second ranges of interest are defined based on the estimated systolic and diastolic blood pressures, respectively. The method includes deflating the cuff at a first deflation rate until the first range of interest is reached and measuring the systolic blood pressure while deflating the cuff at a second deflation rate through the first range of interest. The second deflation rate is less than the first deflation rate. The method includes deflating the cuff at the first deflation rate until the second range of interest is reached, and measuring the diastolic blood pressure while deflating the cuff at the second deflation rate through the second range of interest.

### CROSS-REFERENCE TO RELATED APPLICATION

This application claims the benefit of U.S. Provisional Application No. 63/677,477, filed Jul. 31, 2024, the disclosure of which is hereby incorporated by reference in its entirety.

### BACKGROUND

Blood pressure measurement is a crucial aspect of assessing cardiovascular health and is typically performed using a device called a sphygmomanometer. This instrument consists of an inflatable cuff that is wrapped around the upper arm and a pressure gauge that measures the pressure in the arteries as the cuff is inflated and then slowly released. The measurement is expressed in two numbers: systolic pressure (the pressure in the arteries when the heart contracts) and diastolic pressure (the pressure when the heart is at rest between beats).

The process for measuring blood pressure begins by inflating the cuff to a level above the expected systolic pressure, which temporarily stops blood flow in the artery. As the cuff pressure is slowly released, a healthcare provider listens for the return of blood flow using a stethoscope placed over the artery (known as the auscultatory method) or observes an electronic display (known as the oscillometric method). The point at which the sound of blood flow begins (Korotkoff sounds) or the oscillations cease indicates the systolic pressure, and the point where the sounds disappear completely indicates the diastolic pressure.

Blood pressure measurement is used to assess cardiovascular health, diagnose conditions such as hypertension (high blood pressure), and monitor the effectiveness of treatments. Normal blood pressure readings are typically below 120/80 mmHg (millimeters of mercury). Elevated or abnormal readings may indicate hypertension, which is a significant risk factor for heart disease, stroke, and other health problems. Regular monitoring of blood pressure helps healthcare providers make informed decisions regarding lifestyle modifications, medication management, and overall cardiovascular health maintenance for their patients.

### SUMMARY

In general terms, the present disclosure relates to measuring blood pressure. In one possible configuration, estimates of systolic and diastolic blood pressures are determined during inflation of a cuff, and the estimates are used for guiding measurements of the systolic and diastolic blood pressures during deflation of the cuff. Various aspects are described in this disclosure, which include, but are not limited to, the following aspects.

One aspect relates to a method of measuring blood pressure, the method comprising: estimating systolic and diastolic blood pressures while inflating a cuff wrapped around an extremity of a patient, the estimates of the systolic and diastolic blood pressures being based on waveform oscillations of pressure detected from the cuff; defining first and second ranges of interest based on the estimated systolic and diastolic blood pressures, respectively; deflating the cuff at a first deflation rate until the first range of interest is reached; measuring the systolic blood pressure while deflating the cuff at a second deflation rate through the first range of interest, the second deflation rate being less than the first deflation rate; deflating the cuff at the first deflation rate until the second range of interest is reached; and measuring the diastolic blood pressure while deflating the cuff at the second deflation rate through the second range of interest.

Another aspect relates to a system for measuring blood pressure, comprising: a cuff configured to be wrapped around an extremity of a patient; and a blood pressure monitor connected to the cuff via a tubing, the blood pressure monitor including: a pump for inflating the cuff; one or more pressure release valves for deflating the cuff; one or more sensors for measuring data from the cuff; and a controller including a processing circuitry having non-transitory computer readable storage media storing instructions which, when executed by the processing circuitry, cause the controller to: estimate systolic and diastolic blood pressures while inflating the cuff based on waveform oscillations of pressure detected from the cuff by the one or more sensors; define first and second ranges of interest based on the estimated systolic and diastolic blood pressures, respectively; deflate the cuff at a first deflation rate until the first range of interest is reached; measure the systolic blood pressure while deflating the cuff at a second deflation rate through the first range of interest, the second deflation rate being less than the first deflation rate; deflate the cuff at the first deflation rate until the second range of interest is reached; and measure the diastolic blood pressure while deflating the cuff at the second deflation rate through the second range of interest.

Another aspect relates to a blood pressure monitor, comprising: a pump for inflating a cuff configured to be wrapped around an extremity of a patient; one or more pressure release valves for deflating the cuff; one or more sensors for measuring data from the cuff; and a controller including a processing circuitry having non-transitory computer readable storage media storing instructions which, when executed by the processing circuitry, cause the controller to: estimate systolic and diastolic blood pressures while inflating the cuff based on waveform oscillations of pressure detected from the cuff by the one or more sensors; define first and second ranges of interest based on the estimated systolic and diastolic blood pressures, respectively; deflate the cuff at a first deflation rate until the first range of interest is reached; measure the systolic blood pressure while deflating the cuff at a second deflation rate through the first range of interest, the second deflation rate being less than the first deflation rate; deflate the cuff at the first deflation rate until the second range of interest is reached; measure the diastolic blood pressure while deflating the cuff at the second deflation rate through the second range of interest; wherein the one or more sensors include a pressure sensor, and the estimates of the systolic and diastolic blood pressures are based on waveform oscillations of pressure detected by the pressure sensor while the cuff is being inflated.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 schematically illustrates a system for measuring blood pressure.

FIG. 2 illustrates an isometric view of an example of the system of FIG. 1.

FIG. 3 illustrates a detailed view of the example of the system shown in FIG. 2.

FIG. 4 schematically illustrates an example of a blood pressure measurement application that can be performed by the system of FIG. 1.

FIG. 5 illustrates phases of inflating and deflating a cuff in accordance with the operations of the blood pressure measurement application of FIG. 4.

FIG. 6 schematically illustrates an example of a controller that can be used to implement aspects of the blood pressure measurement system of FIG. 1.

### DETAILED DESCRIPTION

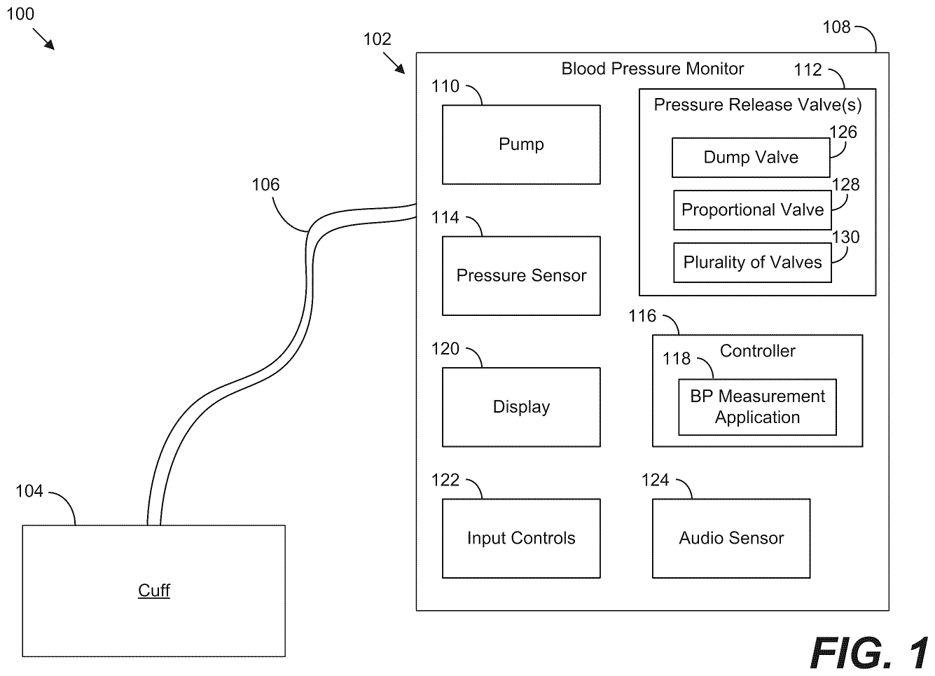

FIG. 1 schematically illustrates a system 100 for measuring blood pressure. As will be described in more detail, the system 100 is configured to perform a new method for measuring systolic and diastolic blood pressures that reduces the time for completing these blood pressure measurements while also ensuring a high fidelity of the blood pressure measurements.

As shown in FIG. 1, the system 100 includes a blood pressure monitor 102 connected to a cuff 104 via a tubing 106. The cuff 104 is configured to be wrapped around an extremity of a patient such as the left arm or the right arm of the patient. Additional anatomical body parts around which the cuff 104 can be wrapped for obtaining blood pressure measurements in accordance with the method described herein are possible.

The cuff 104 is an inflatable sleeve. The tubing 106 permits fluid interconnection between an interior of the inflatable sleeve and one or more components of the blood pressure monitor 102. The cuff 104 can share aspects of the inflatable sleeve described in U.S. Pat. No. 6,578,428, entitled Blood Pressure Measuring Apparatus, issued Jun. 17, 2003, the contents of which are herein incorporated by reference in their entirety.

The blood pressure monitor 102 includes a housing 108 having a pump 110 for inflating the cuff 104, one or more pressure release valves 112 for deflating the cuff 104, and one or more sensors for measuring data from the cuff 104. In the example shown in FIG. 1, the one or more sensors include a pressure sensor 114 that measures waveform oscillations of pressure within the cuff 104 while the cuff 104 is being inflated by the pump 110. The one or more sensors further include an audio sensor 124 that detects audio data within the cuff 104 while the cuff 104 is being deflated by the one or more pressure release valves 112.

The waveform oscillations of pressure that are captured by the pressure sensor 114 when the cuff 104 is being inflated are used to estimate the systolic and diastolic blood pressures from which ranges of interest are determined for measuring the systolic and diastolic blood pressures. The waveform oscillations can more quickly estimate the systolic and diastolic blood pressures than auscultatory methods which monitor for presence and absence of Korotkoff sounds. The ranges of interest are used to guide high fidelity measurements of the systolic and diastolic blood pressures by the audio sensor 124, which reduces the time for obtaining such measurements over traditional auscultatory methods of blood pressure measurement.

The audio data captured by the audio sensor 124 is used to detect the Korotkoff sounds which occur when the systolic blood pressure of the patient exceeds the pressure applied by the cuff 104, and the Korotkoff sounds cease when the diastolic blood pressure is less than the pressure applied by the cuff 104. Accordingly, the audio data captured by the audio sensor 124 when the cuff 104 is being deflated is used by the system 100 to measure the systolic and diastolic blood pressures of the patient. The systolic and diastolic blood pressure measurements determined from the audio data captured by the audio sensor 124 can have a higher degree of fidelity than the systolic and diastolic blood pressures estimates determined by the waveform oscillations of pressure detected by the pressure sensor 114 when the cuff 104 is being inflated.

As further shown in FIG. 1, the blood pressure monitor 102 can further include a display 120 such as to display the systolic and diastolic blood pressure measurements and other outputs determined by the system 100. The blood pressure monitor 102 also includes input controls 122 that can be used to receive inputs from a user of the system 100 such as to initiate a blood pressure measurement. In some examples, the display 120 is a touchscreen such that the input controls 122 can be implemented on the display 120.

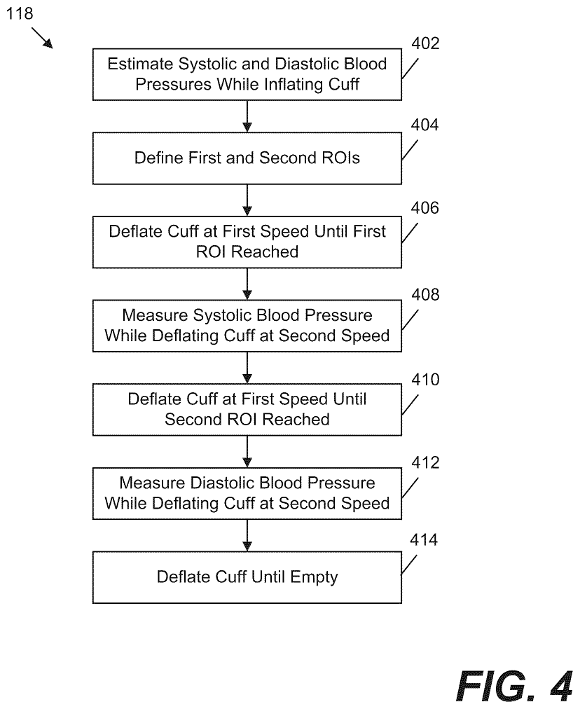

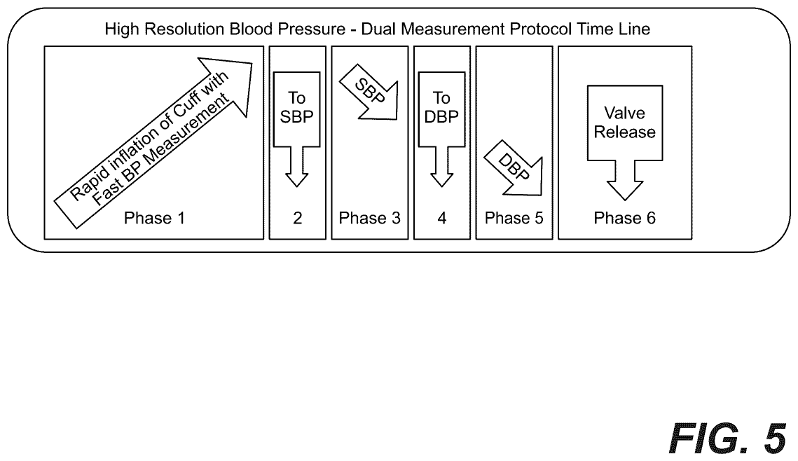

The blood pressure monitor 102 further includes a controller 116 that includes a blood pressure measurement application 118 installed thereon. The controller 116 includes a processing circuitry having non-transitory computer readable storage media storing instructions which, when executed by the processing circuitry, cause the controller 116 to execute the blood pressure measurement application 118 by controlling the pump 110, the one or more pressure release valves 112, the pressure sensor 114, and the audio sensor 124. The blood pressure measurement application 118 is described below in more detail with reference to FIGS. 4 and 5.

The one or more pressure release valves 112 can include a dump valve 126 (also known as a blowoff valve or a compressor bypass valve) that can be controlled by the controller 116 to deflate the cuff 104 in accordance with the blood pressure measurement application 118. In such examples, the dump valve 126 deflates the cuff 104 at a constant rate of deflation.

In alternative examples, the one or more pressure release valves 112 can include a proportional valve 128 that can be controlled by the controller 116 to deflate the cuff 104 in accordance with the blood pressure measurement application 118. In such examples, the proportional valve 128 can be controlled to deflate the cuff 104 at variable rates of deflation.

In further alternative examples, the one or more pressure release valves 112 can include a plurality of valves 130 that each have a different orifice size. Different valves or different combinations of valves can be opened by the controller 116 to deflate the cuff 104 as needed by the blood pressure measurement application 118. For example, a first valve or a first combination of valves can be opened to deflate the cuff 104 at a first deflation rate, and a second valve or a second combination of valves can be opened to deflate the cuff 104 at a second deflation rate. The deflation rate is the speed at which the cuff deflates measured in mmHg/second. A higher deflation rate means that the cuff deflates more quickly, while a lower deflation rate means that the cuff deflates less quickly.

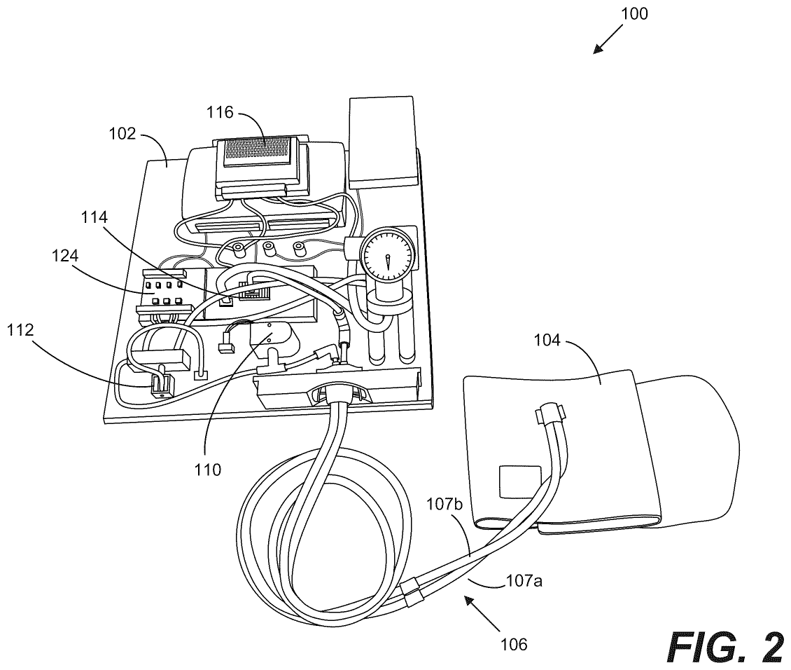

FIG. 2 illustrates an isometric view of an example of the system 100. In this example, the housing 108 is partially removed to show the internal components of the blood pressure monitor 102. As shown in FIG. 2, the cuff 104 is connected via the tubing 106 to the pump 110, the one or more pressure release valves 112, the pressure sensor 114, the audio sensor 124. In this example, the controller 116 is within the housing 108 of the blood pressure monitor 102.

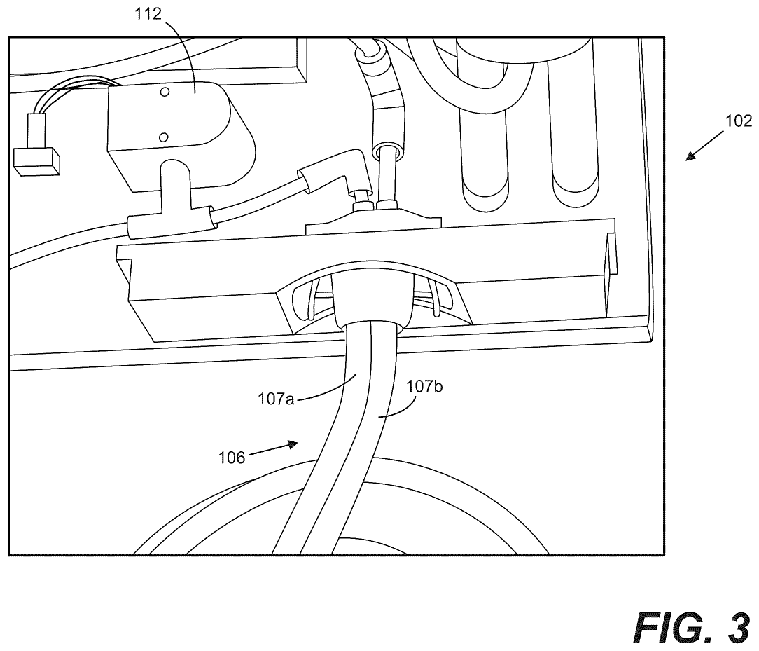

FIG. 3 illustrates a detailed view of the system 100 shown in FIG. 2. As shown in FIG. 3, the tubing 106 is a dual-lumen tube have first and second lumens 107a, 107b that are separated from one another. The dual-lumen design allows the pump 110 and the one or more pressure release valves 112 to be connected to the first lumen 107a of the tubing 106, while the one or more sensors including the pressure sensor 114 and the audio sensor 124 to be connected to the second lumen 107b of the tubing 106 to isolate noise from the pump 110 and the one or more pressure release valves 112 to mitigate and/or eliminate the influence of the noise on the data collected by the pressure sensor 114 and the audio sensor 124.

FIG. 4 schematically illustrates an example of the blood pressure measurement application 118 that can be performed by the system 100. As will be described in more detail below, the blood pressure measurement application 118 reduces the amount of time to acquire systolic and diastolic blood pressure measurements while maintaining high resolution accuracy of these blood pressure measurements, and thereby improving clinical workflows.

As shown in FIG. 4, the blood pressure measurement application 118 includes an operation 402 of estimating systolic and diastolic blood pressures while inflating the cuff 104 wrapped around an extremity of a patient. Operation 402 includes estimating the systolic and diastolic blood pressures based on waveform oscillations of pressure detected within the cuff 104 while the cuff 104 is being inflated around the extremity.

FIG. 5 illustrates phases of inflating and deflating the cuff 104 in accordance with the operations of the blood pressure measurement application 118. In this example, operation 402 of the blood pressure measurement application 118 occurs during phase one which includes rapidly inflating the cuff 104 and simultaneously estimating the systolic and diastolic blood pressures.

The cuff 104 is inflated at an inflation rate of about 8 mmHg/second to about 12 mmHg/second during phase one (i.e., operation 402). In some examples, the cuff 104 is inflated at an inflation rate of about 10 mmHg/second during phase one (i.e., operation 402).

The rapid inflation of the cuff 104 during phase one can cause the pump 110 to generate noise that can interfere with the audio data captured from the cuff 104 by the audio sensor 124. Further, the rapid inflation of the cuff 104 (e.g., about 10 mmHg/second) is too fast for an accurate detection of the Korotkoff sounds from the audio data captured by audio sensor 124. Thus, the blood pressure measurement application 118 can use the waveform oscillations of pressure inside the cuff 104 captured by the pressure sensor 114 to estimate the systolic and diastolic blood pressures when inflating the cuff 104 instead of using the audio data captured by the audio sensor 124 as is done by traditional blood pressure measuring devices.

Referring back to FIG. 4, the blood pressure measurement application 118 includes an operation 404 of defining first and second ranges of interest based on the systolic and diastolic blood pressures, respectively, that are estimated in operation 402. The first and second ranges of interest are ranges of millimeters of mercury (mmHg). In some examples, the first range of interest is about 5 mmHg above and about 5 mmHg below the systolic blood pressure estimated in operation 402. In some examples, the second range of interest is about 5 mmHg above and about 5 mmHg below the diastolic blood pressure estimated in operation 402.

The blood pressure measurement application 118 includes an operation 406 of deflating the cuff 104 at a first deflation rate until the first range of interest (determined in operation 404) is reached. In some examples, the first deflation rate is about 5 mmHg/second to about 8 mmHg/second. Referring now to FIG. 5, operation 406 of the blood pressure measurement application 118 occurs during phase two which includes rapidly deflating the cuff 104 at the first deflation rate.

Referring back to FIG. 4, the blood pressure measurement application 118 includes an operation 408 of measuring the systolic blood pressure while deflating the cuff 104 at a second deflation rate through the first range of interest. The second deflation rate for deflating the cuff 104 in operation 408 is less than the first deflation rate for deflating the cuff 104 in operation 406 (i.e., the cuff deflates at a slower pace or speed). In some examples, the second deflation rate is about 3 mmHg/second. The first deflation rate for deflating the cuff 104 reduces the time for capturing the systolic blood pressure measurement, while the second deflation rate for deflating the cuff 104 provides finer precision for measuring the systolic blood pressure.

In some examples, deflation of the cuff 104 is controlled to have the first deflation rate or the second deflation rate by opening the dump valve 126 of the one or more pressure release valves 112 and operating the pump 110 to counterbalance the opening of the dump valve 126. For example, operating the pump 110 to inject air at a lower pressure such that there is less counterbalance of the opening of the damp valve provides the first deflation rate of deflation, whereas operating the pump 110 to inject air at a higher pressure such that there is more counterbalance of the opening of the damp valve provides the second deflation rate of deflation which is less than the first deflation rate.

In another example, deflation of the cuff 104 is controlled to have the first deflation rate or the second deflation rate by adjusting an aperture of the proportional valve 128. For example, the aperture of the proportional valve 128 is opened to have a larger size to provide the first deflation rate of deflation, whereas the aperture of the proportional valve 128 is opened to have a smaller size to provide the second deflation rate of deflation which is less than the first deflation rate of deflation.

In yet another example, the deflation of the cuff 104 is controlled to have the first deflation rate or the second deflation rate by opening a first valve or a first combination of valves of the plurality of valves 130, and opening a second valve or a second combination of valves of the plurality of valves 130. The first valve can have a larger aperture size than the second valve such that when the first valve is opened, the deflation rate of the cuff 104 is higher than when the second valve is opened. Alternatively, the first combination of valves can have a combined orifice size that is greater than a combined orifice size of the second combination of valves such that when the first combination of valves is opened, the deflation rate of the cuff 104 is higher than when the second combination of valves is opened.

Operation 408 includes measuring the systolic blood pressure based on the audio data captured from the cuff 104 by the audio sensor 124 while the cuff 104 is being deflated at the second deflation rate through the first range of interest. For example, operation 408 includes deflating the cuff 104 at the second deflation rate and simultaneously monitoring for the Korotkoff sounds for measurement of the systolic blood pressure. As described above, the lower second deflation rate for deflating the cuff 104 provides finer precision for measuring the systolic blood pressure than the first deflation rate that rapidly deflates the cuff 104.

Referring now to FIG. 5, operation 408 of the blood pressure measurement application 118 occurs during phase three. As shown in FIG. 5, the deflation rate of the cuff 104 during phase three is less than the deflation rate of the cuff 104 during phase two.

Referring back to FIG. 4, the blood pressure measurement application 118 includes an operation 410 of deflating the cuff 104 at the first deflation rate until the second range of interest is reached. As described above, the first deflation rate is higher than the second deflation rate which reduces the time for capturing the diastolic blood pressure measurement. Operation 410 of the blood pressure measurement application 118 occurs during phase four in FIG. 5 which includes rapidly deflating the cuff 104 at the first deflation rate until the second range of interest is reached (i.e., where the diastolic blood pressure estimated in operation 402 is located).

Referring back to FIG. 4, the blood pressure measurement application 118 includes an operation 412 of measuring the diastolic blood pressure while deflating the cuff at the second deflation rate through the second range of interest. The second deflation rate for deflating the cuff 104 in operation 412 is less than the first deflation rate for deflating the cuff 104 in operation 410. Operation 412 includes measuring the diastolic blood pressure based on the audio data captured from the cuff 104 by the audio sensor 124 while the cuff 104 is being deflated at the second deflation rate through the second range of interest. For example, operation 412 includes deflating the cuff 104 at the second deflation rate and simultaneously monitoring for cessation of the Korotkoff sounds for measurement of the diastolic blood pressure. The second deflation rate for deflating the cuff 104 in operation 412 provides finer precision than the first deflation rate for measuring the diastolic blood pressure.

Referring now to FIG. 5, operation 412 of the blood pressure measurement application 118 occurs during phase five. As shown in FIG. 5, the deflation rate during phase five is less than the deflation rate during phase four.

Referring back to FIG. 4, the blood pressure measurement application 118 includes an operation 414 of deflating the cuff 104 until it is empty. Operation 414 can include deflating the cuff 104 at the first deflation rate (which is higher than the second deflation rate) until remaining air inside the cuff 104 is emptied. In FIG. 5, operation 414 occurs during phase six. As shown in FIG. 5, the deflation rate during phase six is higher than the deflation rate during phase five.

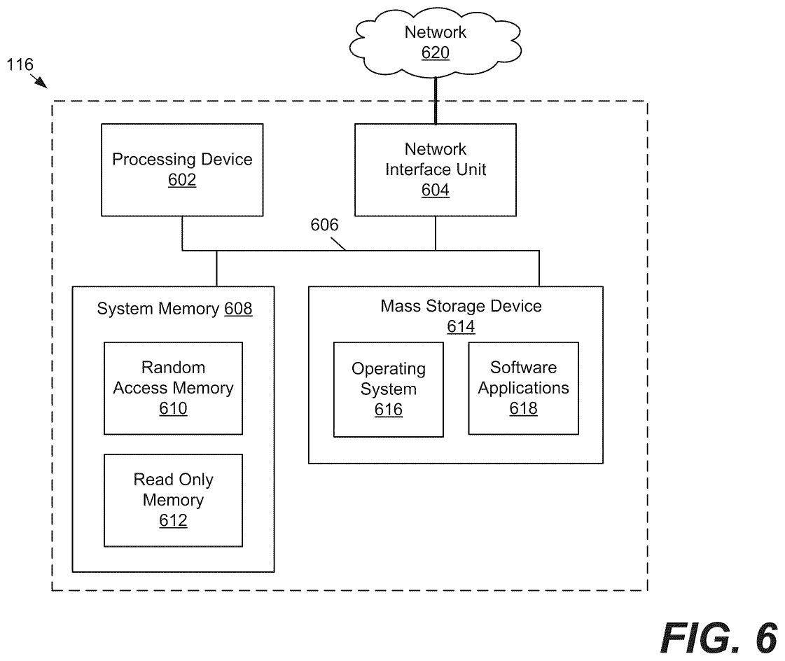

FIG. 6 schematically illustrates an example of the controller 116 that can be used to implement aspects of the system 100 including execution of the blood pressure measurement application 118. The controller 116 includes a processing circuitry having non-transitory computer readable storage media storing instructions which, when executed by the processing circuitry, cause the controller 116 to execute the blood pressure measurement application 118 by controlling the pump 110, the one or more pressure release valves 112, the pressure sensor 114, and the audio sensor 124. In the example of FIG. 6, the controller 116 is shown as including at least one processing device 602, a system memory 608, and a system bus 606 that couples the system memory 608 to the at least one processing device 602.

The controller 116 can operate in a networked environment using logical connections to devices through a network 620. The controller 116 can connect to the network 620 through a network interface unit 604 connected to the system bus 606. The network interface unit 604 can connect to the network 620 using one or more wired connections and protocols such as Ethernet, or using one or more wireless connections and protocols such as Bluetooth and Wi-Fi.

The at least one processing device 602 is an example of a processing unit such as a central processing unit (CPU). The at least one processing device 602 can include one or more CPUs. In some examples, the at least one processing device 602 includes one or more digital signal processors, field-programmable gate arrays, and/or other types of electronic circuits.

The system memory 608 includes a random-access memory (“RAM”) 610 and a read-only memory (“ROM”) 612. Basic input/output logic containing routines to transfer information between elements within the controller 116 is stored in the ROM 612.

The controller 116 can also include a mass storage device 614 that is able to store software instructions and data. The mass storage device 614 is connected to the at least one processing device 602 through a mass storage controller connected to the system bus 606. The mass storage device 614 and its associated computer-readable data storage media provide additional non-volatile, non-transitory storage for the controller 116.

The mass storage device 614 and/or the system memory 608 can store software instructions and data. The software instructions can include an operating system 616 suitable for controlling the operation of the controller 116. The mass storage device 614 and/or the system memory 608 also store software instructions 618, that when executed by the at least one processing device 602, cause the device to provide the functionality discussed herein.

Although the description of computer-readable data storage media contained herein refers to a mass storage device, it should be appreciated by those skilled in the art that computer-readable data storage media can be any available non-transitory, physical device or article of manufacture from which the device can read data and/or instructions. In certain embodiments, the computer-readable storage media comprises entirely non-transitory media. The mass storage device 614 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

The various embodiments described above are provided by way of illustration only and should not be construed to be limiting in any way. Various modifications can be made to the embodiments described above without departing from the true spirit and scope of the disclosure.

What is claimed is:

1. A method of measuring blood pressure, the method comprising:
estimating systolic and diastolic blood pressures while inflating a cuff wrapped around an extremity of a patient, the estimates of the systolic and diastolic blood pressures being based on waveform oscillations of pressure detected from the cuff;
defining first and second ranges of interest based on the estimated systolic and diastolic blood pressures, respectively;
deflating the cuff at a first deflation rate until the first range of interest is reached;
measuring the systolic blood pressure while deflating the cuff at a second deflation rate through the first range of interest, the second deflation rate being less than the first deflation rate;
deflating the cuff at the first deflation rate until the second range of interest is reached; and
measuring the diastolic blood pressure while deflating the cuff at the second deflation rate through the second range of interest.

2. The method of claim 1, wherein the first range of interest is about 5 mmHg above and about 5 mmHg below the estimate of systolic blood pressure, and the second range of interest is about 5 mmHg above and about 5 mmHg below the estimate of diastolic blood pressure.

3. The method of claim 2, wherein the first deflation rate is about 8 mmHg/second, and the second deflation rate is about 3 mmHg/second.

4. The method of claim 1, wherein measuring the systolic blood pressure is based on audio data from the cuff while being deflated at the second deflation rate through the first range of interest, and measuring the diastolic blood pressure is based on the audio data from the cuff while being deflated at the second deflation rate through the second range of interest.

5. The method of claim 4, wherein estimating the systolic and diastolic blood pressures while inflating the cuff is based on waveform oscillations of pressure captured by a pressure sensor.

6. The method of claim 5, further comprising:
inflating the cuff at an inflation rate of about 8 mmHg/second to about 12 mmHg/second when estimating systolic and diastolic blood pressures.

7. A system for measuring blood pressure, the system comprising:
a cuff configured to be wrapped around an extremity of a patient; and
a blood pressure monitor connected to the cuff via a tubing, the blood pressure monitor including:
a pump for inflating the cuff;
one or more pressure release valves for deflating the cuff;
one or more sensors for measuring data from the cuff; and
a controller including a processing circuitry having non-transitory computer readable storage media storing instructions which, when executed by the processing circuitry, cause the controller to:
estimate systolic and diastolic blood pressures while inflating the cuff based on waveform oscillations of pressure detected from the cuff by the one or more sensors;
define first and second ranges of interest based on the estimated systolic and diastolic blood pressures, respectively;
deflate the cuff at a first deflation rate until the first range of interest is reached;
measure the systolic blood pressure while deflating the cuff at a second deflation rate through the first range of interest, the second deflation rate being less than the first deflation rate;
deflate the cuff at the first deflation rate until the second range of interest is reached; and
measure the diastolic blood pressure while deflating the cuff at the second deflation rate through the second range of interest.

8. The system of claim 7, wherein the first range of interest is about 5 mmHg above and about 5 mmHg below the estimate of systolic blood pressure, and the second range of interest is about 5 mmHg above and about 5 mmHg below the estimate of diastolic blood pressure.

9. The system of claim 8, wherein the first deflation rate is about 8 mmHg/second, and the second deflation rate is about 3 mmHg/second.

10. The system of claim 7, wherein the one or more sensors include an audio sensor, and the measurements of the systolic and diastolic blood pressures are based on audio data detected by the audio sensor while the cuff is being deflated at the second deflation rate through the first and second ranges of interest, respectively.

11. The system of claim 10, wherein the one or more sensors include a pressure sensor, and estimate the systolic and diastolic blood pressures while inflating the cuff is based on waveform oscillations of pressure captured by the pressure sensor.

12. The system of claim 7, wherein deflation of the cuff is controlled to have the first deflation rate or the second deflation rate by opening a dump valve of the one or more pressure release valves and operating the pump to counterbalance the opening of the dump valve.

13. The system of claim 7, wherein the one or more pressure release valves include a proportional valve to control deflation of the cuff to have the first deflation rate or the second deflation rate.

14. The system of claim 7, wherein the one or more pressure release valves include a plurality of valves, and a first valve or a first combination of valves of the plurality of valves is opened to deflate the cuff at the first deflation rate, and a second valve or a second combination of valves of the plurality of valves is opened to deflate the cuff at the second deflation rate.

15. The system of claim 7, wherein the pump and the one or more pressure release valves are connected to a first lumen of the tubing, and the one or more sensors are connected to a second lumen of the tubing to isolate noise from the pump and the one or more pressure release valves.

16. A blood pressure monitor, comprising:
a pump for inflating a cuff configured to be wrapped around an extremity of a patient;
one or more pressure release valves for deflating the cuff;
one or more sensors for measuring data from the cuff; and
a controller including a processing circuitry having non-transitory computer readable storage media storing instructions which, when executed by the processing circuitry, cause the controller to:
estimate systolic and diastolic blood pressures while inflating the cuff based on waveform oscillations of pressure detected from the cuff by the one or more sensors;
define first and second ranges of interest based on the estimated systolic and diastolic blood pressures, respectively;
deflate the cuff at a first deflation rate until the first range of interest is reached;
measure the systolic blood pressure while deflating the cuff at a second deflation rate through the first range of interest, the second deflation rate being less than the first deflation rate;
deflate the cuff at the first deflation rate until the second range of interest is reached;
measure the diastolic blood pressure while deflating the cuff at the second deflation rate through the second range of interest;
wherein the one or more sensors include a pressure sensor, and the estimates of the systolic and diastolic blood pressures are based on waveform oscillations of pressure detected by the pressure sensor while the cuff is being inflated.

17. The blood pressure monitor of claim 16, wherein the one or more sensors further include an audio sensor, and the measurements of the systolic and diastolic blood pressures are based on audio data detected by the audio sensor while the cuff is being deflated at the second deflation rate through the first and second ranges of interest, respectively.

18. The blood pressure monitor of claim 16, wherein deflation of the cuff is controlled to have the first deflation rate or the second deflation rate by opening a dump valve of the one or more pressure release valves and operating the pump to counterbalance the opening of the dump valve.

19. The blood pressure monitor of claim 16, wherein the one or more pressure release valves include a proportional valve to control deflation of the cuff at the first deflation rate or the second deflation rate.

20. The blood pressure monitor of claim 16, wherein the one or more pressure release valves include a plurality of valves, and a first valve or a first combination of valves of the plurality of valves is opened to deflate the cuff at the first deflation rate, and a second valve or a second combination of valves of the plurality of valves is opened to deflate the cuff at the second deflation rate.
